(19) United States
(12) Patent Application Publication
(10) Pub. No.: US 2026/0232544 A1
(43) Pub. Date: Aug. 13, 2026

(54) FILM-BASED SKIN CARE PRODUCTS FOR TOPICAL APPLICATION

(71) Applicant: Reg Macquarrie, Bangkok (TH)

(72) Inventor: Reg Macquarrie, Bangkok (TH)

(21) Appl. No.: 19/475,277

(22) PCT Filed: Apr. 12, 2024
(86) PCT No.: PCT/CA2024/050473

§ 371 Date: Oct. 14, 2025

Related U.S. Application Data
(60) Provisional application No. 63/495,919, filed on Apr. 13, 2023.

(51) Int. Cl. A61K 8/02; A61Q 17/00; A61Q 19/08

(52) U.S. Cl. CPC A61K 8/0212; A61Q 17/00; A61Q 19/08; A61K 2800/244; A61K 2800/54; A61K 2800/5922

(57) ABSTRACT

A skin care product such as a cosmetic face mask is disclosed for topical application to a user's skin. The product has at least a base layer and a functional layer. The base layer is a flexible, biodegradable film including a biodegradable polymer matrix and a water-soluble plasticizer. The functional layer is applied to the base layer and includes one or more active ingredients to provide a cosmetic or medicinal benefit when applied to the user's skin. The product may also include a non-aqueous third layer applied on the surface of the functional layer which is opposite to the base layer. The third layer includes one or more oils, and is absorbed or dispersed upon contact with the user's skin, to expose the underlying functional layer to the skin surface. After use, the skin care product can be disposed of by dissolving in water or composting.

### FIELD

The present disclosure relates to skin care products for topical application to the skin of a user. More specifically, the disclosure relates to skin care products having a base layer comprised of a biodegradable film, and having one or more active ingredients which provide a cosmetic and/or medicinal benefit when applied to the user's skin. The skin care product may be in the form of a face mask for direct application to the user's face, or in the form of a patch or film for application to other areas of the user's skin.

### BACKGROUND

Healthy skin is a common goal globally. Consumers looks for methods to improve the appearance and feel of their skin and address skin flaws. Cosmetic face masks are commonly used for this purpose, as an effective method of delivering functional ingredients to the surface of a user's skin. A cosmetic face mask can be customized to provide a specific treatment, such as hydration, cooling, blemish control, wrinkle reduction or skin firming.

Cosmetic face masks are typically made of woven materials such as cotton, fiber, cellulose or pulp. The woven material is pre-cut to the required shape, most commonly being the entire face, and is soaked in various solutions that contain cosmetic compounds, typically referred to as a serum. The woven material is used to transfer and hold the cosmetic compounds against the face for a desired amount of time. Upon completion of the treatment the face mask is discarded in the trash, as the mask is generally made of non-compostable, non-recyclable, non-biodegradable materials. These masks may also be made from synthetic resins that do not biodegrade and thus contribute to plastic waste.

Another issue with conventional cosmetic face masks is that they are inconvenient and messy to use. Because the woven material is merely soaked in the serum, it may not adequately hold the serum, and a portion of the serum may separate from the mask during removal from its package and during application by the user. Therefore, a portion of the serum may remain in the packaging or on the user's hands, and the full benefit of the active components in the serum may not be achieved.

There is a need for improved skin care products for topical application which avoid the above disadvantages.

### SUMMARY

In an embodiment, a skin care product for topical application may comprise a base layer and a functional layer. The base layer may comprise a flexible, biodegradable film, wherein the film comprises a biodegradable polymer matrix and a water-soluble plasticizer. The functional layer may be applied to one surface of the base layer, and may comprise one or more active ingredients to provide a cosmetic and/or medicinal benefit when applied topically to a surface of a user's skin.

In another aspect, the biodegradable polymer matrix may comprise one or more polymeric materials selected from the group comprising polysaccharides, flours, fibrous materials, gums and proteins.

In another aspect, the polymer matrix may comprise about 35-80% of the base layer composition.

In another aspect, the biodegradable polymer matrix may comprise one or more water soluble starches.

In another aspect, the biodegradable polymer matrix may comprise one or more water soluble starches selected from the group comprising potato starch, wheat starch, tapioca starch, corn starch, rice starch, pea starch, bean starch, lentil starch, pulse starch and mung bean starch.

In another aspect, the biodegradable polymer matrix may comprise one or more gums selected from the group comprising carrageenan, konjac, gellan gum, agar, guar gum, locust bean gum, xanthan gum, carboxymethylcellulose and alginates.

In another aspect, the biodegradable polymer matrix may comprise one or more vegetable proteins selected from the group comprising soy protein, rice protein, pea protein, mung bean protein, hemp protein and pumpkin seed protein.

In another aspect, the biodegradable polymer matrix may comprise one or more water-soluble starches and one or more gums, wherein a weight ratio of starch:gum in the biodegradable polymer matrix may be from about 2:1 to about 30:1.

In another aspect, the water-soluble plasticizer may be glycerin, and the base layer may comprise about 10-30% of the plasticizer.

In another aspect, the base layer may comprise about 10% water.

In another aspect, the base layer may have a thickness of about 40-60 microns.

In another aspect, the active ingredients of the functional layer may be one or more of hyaluronic acid, ceramides, aloe vera, menthol, chamomile, pentavitin, niacinamide, peppermint oil, witch hazel, salicylic acid, tea tree oil, colloidal oatmeal, hydrocortisone, pullulan, caffeine, vitamin C, Paeonia suffruticosa root extract, Centella asiatica extract, and citrus extracts.

In another aspect, the skin care product may be a face mask, and the active ingredients may be selected from one or more of hyaluronic acid, ceramides, salicylic acid and natural extracts.

In another aspect, the skin care product may be a cooling film, and the active ingredients may be selected from one or more of menthol, aloe vera and natural extracts.

In another aspect, the skin care product may be an insect bite film, and the active ingredients may be selected from one or more of colloidal oatmeal, hydrocortisone and natural extracts.

In another aspect, the skin care product may be for skin tightening, and the active ingredients may be selected from one or more of hydroxypropyl methylcellulose, pullulan, hydrolyzed collagen, elastin, and natural extracts.

In another aspect, the functional layer may include one or more film forming compounds selected from a biodegradable polymer matrix and a water-soluble plasticizer.

In another aspect, the one or more film forming compounds may be selected from the group comprising glycerin, water-soluble starch and alginate.

In another aspect, the functional layer may have a thickness of about 35-40 microns.

In another aspect, the skin care product may include a third layer which is provided on top of the functional layer, such that the functional layer is sandwiched between the base layer and the functional layer, wherein the third layer may have a non-aqueous composition.

In another aspect, the third layer may comprise one or more oils selected from the group comprising coconut oil, avocado oil, and jojoba oil.

In another aspect, the third layer may have a thickness of about 10-15 microns.

### BRIEF DESCRIPTION OF THE DRAWINGS

The invention will now be described, by way of example only, with reference to the accompanying drawings in which:

FIG. 1 is a schematic drawing showing the layers of a skin care product according to the present disclosure; and

FIG. 2 illustrates an example of a skin care product in the form of a cosmetic face mask.

### DETAILED DESCRIPTION

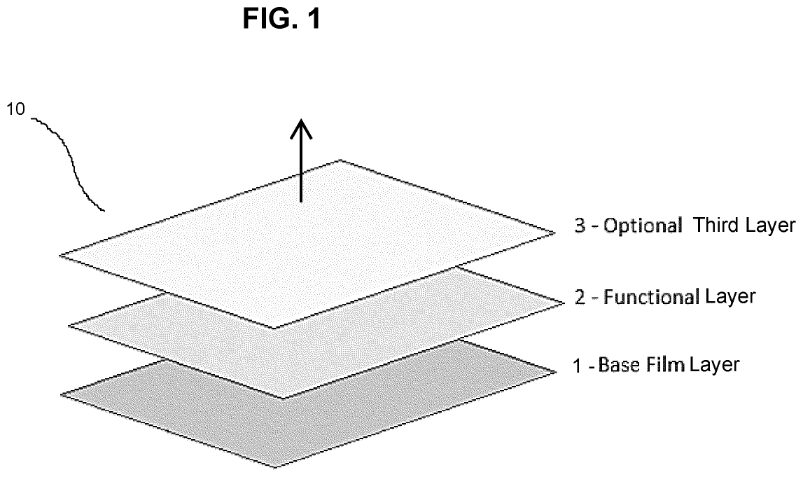

The embodiments described herein relate to skin care products for topical application to a user's skin. As illustrated in FIG. 1, the skin care products 10 generally comprise a base layer 1 and a functional layer 2 applied to one surface of the base layer 1.

Unless otherwise stated, all percentages and ratios disclosed herein are by weight.

The base layer comprises a flexible, biodegradable film, wherein the film generally comprises a biodegradable polymer matrix, a water-soluble plasticizer, water and other ingredients. The major components of the base layer are generally vegetable-based hydrocolloids derived from natural and renewable sources.

The biodegradable polymer matrix comprises one or more polymeric materials selected from the group comprising polysaccharides, flours, fibrous materials, gums and proteins. The polymeric materials are generally vegetable-based, and the polymer matrix typically comprises about 35-80% of the base layer composition. More typically, the polymer matrix may comprise 40-75% of the base layer composition.

Suitable polysaccharides are water soluble and include modified or unmodified (native) starches, which may be selected from one or more members of the group comprising potato starch, wheat starch, tapioca starch, corn starch, rice starch, pea starch, bean starch, lentil starch, pulse starch and mung bean starch. Rice starch and/or tapioca starch are most commonly used in the example embodiments of the base layer.

Suitable flours may be selected from one or more members of the group comprising rice flour, wheat flour, bean flour and corn flour.

Examples of fibrous materials include vegetable fibres such as pea fibre, rice fibre, tapioca fibre, wheat fibre and potato fibre, and may include cellulose.

Suitable gums may be selected from one or more members of the group comprising carrageenan, konjac, gellan gum, agar, guar gum, locust bean gum, xanthan gum, carboxymethylcellulose and alginates. Carrageenan is most commonly used in the example embodiments of the base layer.

The proteins may be derived from natural sources, and may be of animal or vegetable origin. Suitable vegetable proteins may be selected from one or more members of the group comprising soy protein, rice protein, pea protein, mung bean protein, hemp protein, pumpkin seed protein, and like vegetable proteins.

In some example embodiments, the polymer matrix of the base layer composition is comprised of one or more starches and a gum such as carrageenan. In such embodiments, the polymer matrix is predominantly starch-based. For example, a weight ratio of starch:gum may be in the range from about 2:1 to about 30:1, or on a weight percentage basis, the starch may make up about 70-98% of the polymer matrix components.

Suitable plasticizers for use in the base layer may be selected from one or more members of the group comprising natural humectants, such as glycerin (also known as glycerol), sorbitol, hyaluronic acid, sodium lactate and honey. Propylene glycol may be used as a plasticizer in some embodiments. Most of the example embodiments use glycerin as the plasticizer, wherein the glycerin may be naturally sourced, for example from palm. The plasticizer typically makes up about 10-30% of the base layer composition, more typically about 15-25%.

The water content of the base layer is typically from about 5-15%, for example about 10%. This is the water content of the film after drying.

The base layer film composition may include one or more other ingredients such as oils, fragrances, flavours and colours. Suitable oils include coconut oil and palm oil, which may be included in the base film in amounts ranging from about 2-7%.

The base layer film may be provided in a wide range of thicknesses, with the dry film thickness of the base layer being about 10-150 microns or about 10-120 microns, typically about 25-70 microns, and more typically about 40-60 microns.

The base layer film can be manufactured by solution casting or by extrusion.

In solution casting, some or all of the above-mentioned base film components such as gums, starches, oils, fibers, proteins and plasticizers are mixed with water at increasing temperatures to form a uniform solution. The solution typically has a target viscosity of about 6,000-15,000 cps. The solution may also include colour, flavour and/or fragrance as required. The solution is cast on a continuous belt or foil and dried to the required moisture content and a uniform thickness. The film is formulated to have tensile strength and elongation properties as required for the intended application, and may be slit, cut and/or stamped to any required size and shape.

Exemplary values of tensile strength and elongation for a facemask base film are provided in the table below, both before drying (“wet”) and after drying (“dry”). One can see that these mechanical properties are improved by drying the film.

MECHANICAL PROPERTIES OF FACE MASK BASE FILM

| | |
|---|---|
| DRY | WET |

| Tensile | Elongation | Tensile | Elongation |
|---|---|---|---|
| strength (mPa) | (%) | strength (mPa) | (%) |
| 13.0 ± 0.38 | 21.0 ± 2.2 | 1.9 ± 0.29 | 5.9 ± 0.45 |

In extrusion, the dry film components are pre-mixed to form a dry blend which is fed into the mixer of the extruder by either a one-step or two-step process.

In the one step process, the dry blend is fed into the extruder and mixed with liquid film components such as water and/or glycerin through various heating zones and the required screw speed to form a molten composition. The molten composition is then fed through a film die and the extruded base film is dried to the required moisture content. The dried base film can be further processed into rolls or sheets as needed.

In the two-step process, the film components are initially premixed and then fed into the extruder and mixed under a specified set of conditions, including temperature and screw speed to form a molten composition which is extruded through a tubular die to form a consistent tube that is then cut into smaller sizes, commonly referred to as pellets.

The pellets are then fed into the extruder again, melted and pushed through a flat or tubular die of suitable size to achieve the desired film thickness, and the extruded base film is dried to the required moisture content. The dried base film can be further processed into rolls or sheets as needed.

The selection of a one step or two step extrusion process is dependent the type of compounds being utilized and their hydration rates.

Once the base film is prepared and dried to a suitable moisture content, it may then be provided with a functional layer comprising one or more active ingredients to provide a cosmetic and/or medicinal benefit when applied to a surface of a user's skin. In use, the functional layer faces and is in direct contact with the user's skin, while the base film layer faces away from the user's skin. The thickness of the functional layer may be about 25-50 microns, and more typically about 35-40 microns.

The active ingredients of the functional layer may provide one or more benefits to the user's skin, such as hydration, cooling, soothing, brightening, blemish control, relief from insect bites or stings, skin tightening or firming, and cellulite control. These effects can be cosmetic and/or medicinal. Exemplary formulations for various functional layers are provided in the Examples.

To provide the range of benefits mentioned above, a wide variety of active ingredients may be provided in the functional layer. For example, the active ingredients can include hyaluronic acid, ceramides, aloe vera, menthol, chamomile, pentavitin, niacinamide, peppermint oil, witch hazel, salicylic acid, tea tree oil, colloidal oatmeal, hydrocortisone, pullulan, caffeine, vitamin C, etc. The active ingredients can also include natural extracts such as Paeonia suffruticosa root extract, Centella asiatica extract, citrus extracts, etc.

For example, where the skin care product is for face mask applications, the active ingredients may include one or more of hyaluronic acid, ceramides, salicylic acid and natural extracts.

Where the skin care product is a cooling film, the active ingredients may include one or more of menthol, aloe vera and natural extracts.

Where the skin care product is an insect bite film, the active ingredients may include one or more of colloidal oatmeal, hydrocortisone and natural extracts.

Where the skin care product is for skin tightening, the active ingredients may include one or more of hydroxypropyl methylcellulose, pullulan, hydrolyzed collagen and elastin, and natural extracts.

In some embodiments, the functional layer may be applied to the base film layer in the form of a liquid slurry. The slurry is applied to the base film layer at a required thickness and is then dried. In other embodiments, the functional layer may be applied by roll coating or spray coating onto the base film layer.

The functional layer may additionally include one or more film forming compounds to ensure that the functional layer is smooth, and to adjust the rate of dissolution of the mask upon hydration and application to the skin. These film forming compounds in the functional layer may include any of the above-mentioned film forming compounds of the base layer, i.e. the biodegradable polymer matrix, water-soluble plasticizer, water and other ingredients as described above. For example, the biodegradable polymer matrix may be selected from the group comprising polysaccharides, flours, fibrous materials, gums and proteins. For example, the functional layers may include glycerin, water-soluble starch and alginate.

Other ingredients which may be included in the functional layer include oils, fragrances, flavours and colours, as discussed above.

The skin care product may also include an optional third layer, which may have a non-aqueous composition. The third layer is identified as layer 3 in FIG. 1. In some embodiments, the third layer comprises an oil such as coconut oil, avocado oil, jojoba oil, etc. This third layer is applied on top of the functional layer, such that the functional layer is sandwiched between the base film layer and the functional layer. The third layer therefore comes into direct contact with the user's skin when the skin care product is first applied to the skin, with the arrow in FIG. 1 indicating the direction which faces the user's skin. The third layer is absorbed into the skin and/or disperses, to expose the functional layer to the skin surface.

The presence of a non-aqueous third layer as a surface coating provides one or more benefits, such as: providing ease of handling to avoid loss of the functional layer prior to or during application to the skin; due to its oily nature, allowing the user to manipulate the product for optimal placement onto the desired area of the user's skin; avoiding loss of the functional layer during hydration of the skin care product; and/or further enhancing the intended skin treatment, for example by providing skin hydration properties.

Typically, the optional third layer is typically a lighter, thinner coating than either the functional layer or the base film layer. The third layer may be applied by spraying it onto the exposed surface of the functional layer. To allow the third layer to be quickly absorbed and/or dispersed upon application to the user's skin, the third layer may have a thickness of about 5-20 microns, and more typically about 10-15 microns.

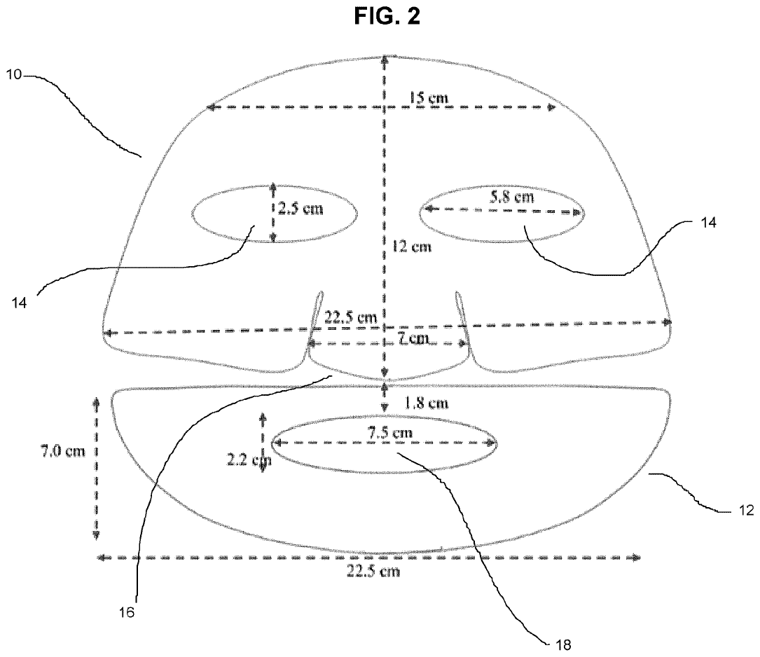

After the layers are formed and dried, the resulting product will typically be in the form of a flat sheet, with the exact size and dimensions typically depending on the manner in which the base film layer was formed. Individual skin care products are then provided by cutting the flat sheet product, and/or or stamping it with one or more dies. For example, as shown in FIG. 2, where the skin care product 10 is a face mask, the flat sheet product is cut or stamped to form one or more pieces defining the outer edges 12 of the face mask, and to define holes for the user's eyes 14, nose 16 and/or mouth 18. The flat sheet product may be cut and/or stamped into a variety of other shapes and sizes, depending on the use of the skin care product. For example, the flat sheet product may be cut and/or stamped into patches of various sizes for application under the eyes or for application to other areas of the body to treat insect bites, blemishes, or provide any other treatments mentioned herein.

After cutting and/or stamping into the desired shape, the skin care product is packaged for sale. Because the skin care product is packaged in dried form, there is a reduced chance that the functional layer and/or any of the active ingredients contained therein will degrade due to lengthy aqueous storage, which can be an issue with conventional “wet” masks. The dry nature of the product also simplifies handling and application by the user, and results in less of the functional layer and/or active ingredients being left behind in the packaging.

Once it is removed from the package by the user, the skin care product may be hydrated briefly in water, before being applied to the user's skin. Hydration may be useful to activate the functional layer and/or to improve flexibility, application and/or adhesion of the skin care product to the user's skin.

While a face mask product according to the present disclosure will typically be hydrated before use, it will be appreciated that hydration may not always be required, and that some skin care products according to the present disclosure may be directly applied to the skin after being removed from the packaging.

Once the skin care treatment is complete, the skin care product is removed by the user and discarded. Due to the biodegradable and/or soluble nature of the ingredients comprising the base film, the skin care product may be disposed of in an ecologically friendly manner, for example, by dissolving it in warm water and washing it down the sink, or by adding it to a compostable waste stream. Unlike conventional cloth-based face masks, for example, a face mask according to the present disclosure leaves behind no long-lasting solid waste, making it more attractive to environmentally conscious consumers. Due to the favourable properties of the skin care product according to the present disclosure, and the selection of natural ingredients for the base layer, functional layer and other optional layers, certain embodiments of the skin care product may be advertised as being vegan, paraben free, sulfate free, mineral oil free, petrolatum free, and/or silicone free.

### EXAMPLES

### Example 1—Base Film Formulations

The following table lists various formulations of the base film layer. All four columns show “dry” formulations, i.e. after drying, with the first column showing the exact composition of an exemplary base film layer, and the other columns showing percentage ranges for the ingredients of the film.

| | Formulation A | Formulation 1 | Formulation 2 | Formulation 3 |
|---|---|---|---|---|
| Ingredients | (%) | (% Range) | (% Range) | (% Range) |
| Water | 10.00 | 5-10 | 5-10 | 5-10 |
| Native Rice starch | 12.26 | 10-12 | 10-12 | 5-10 |
| Glutinous rice starch | 42.78 | 40-50 | 25-30 | 45-55 |
| Coconut oil | 5.03 | 2-7 | 2-7 | 0 |
| Glycerin | 21.56 | 20-25 | 20-25 | 15-20 |
| Carrageenan | 8.38 | 5-10 | 7-15 | 2-5 |

### Example 2—Functional Layer Formulation (Face Mask)

The following table lists the ingredients of a functional layer formulation for a face mask.

This particular functional layer formulation includes glycerin, starch and sodium alginate as film forming ingredients. The other listed ingredients except for colour and fragrance comprise the active ingredients of the functional layer. This functional layer formulation was applied to the first base film formulation listed in the table of Example 1 above, but may be applied to any of the base film formulations disclosed in Example 1.

| Ingredients | % Dry |
|---|---|
| Water | 10.00% |
| Glycerin | 9.59% |
| High Amylose gel starch (Cargill) | 31.47% |
| Sodium alginate (Manucol LD) | 39.63% |
| Hyaluronic acid (High Mw) | 0.42% |
| Paeonia Suffruticosa root extract | 0.29% |
| Centella Asiatica extract | 0.29% |
| Chamomilla recutita flower | 0.15% |
| Pentavitin | 1.46% |
| Color (Turmetic /Beet root) | 5.25% |
| Fragrance (rice milk oil) | 0.29% |
| Niacinamide | 0.29% |
| Peppermint oil | 0.87% |
| Total | 100.00% |

### Example 3—Functional Layer Formulation (Face Mask)

The following table lists the ingredients of a functional layer formulation for a face mask to provide a skin hydrating function.

This particular functional layer formulation includes glycerin, starch and sodium alginate as film forming ingredients. The other listed ingredients except for colour and fragrance comprise the active ingredients of the functional layer. This functional layer formulation was applied to the base film formulation 1 listed in the table of Example 1 above, but may be applied to any of the base film formulations disclosed in Example 1.

| Ingredients | % Dry |
|---|---|
| Water | 5-10 |
| Glycerin | 5-10 |
| Starch | 30-35 |
| Alginate | 35-45 |
| Hyaluronic Acid | 0.4-1.5 |
| Pentavitin | 1-2 |
| Extracts | 1-2 |
| Fragrance, Colour | 5-6 |

### Example 4—Functional Layer Formulation (Face Mask)

The following table lists the ingredients of a functional layer formulation for a face mask to provide a cooling, soothing function.

This particular functional layer formulation includes glycerin, starch and sodium alginate as film forming ingredients. The other listed ingredients except for colour and fragrance comprise the active ingredients of the functional layer. This functional layer formulation was applied to the base film formulation 2 listed in the table of Example 1 above, but may be applied to any of the base film formulations disclosed in Example 1.

| Ingredients | % Dry |
|---|---|
| Water | 5-10 |
| Glycerin | 5-10 |
| Starch | 15-20 |
| Alginate | 35-45 |
| Aloe Vera | 15-20 |
| Menthol | 0.5-1.0 |
| Witch hazel | 0.2-0.5 |
| Extracts | 1-2 |
| Fragrance, Colour | 1-2 |

### Example 5—Functional Layer Formulation (Face Mask)

The following table lists the ingredients of a functional layer formulation for a face mask to provide a skin brightening function.

This particular functional layer formulation includes glycerin, starch and sodium alginate as film forming ingredients. The other listed ingredients except for colour and fragrance comprise the active ingredients of the functional layer. This functional layer formulation was applied to the base film formulation 2 listed in the table of Example 1 above, but may be applied to any of the base film formulations disclosed in Example 1.

| Ingredients | % Dry |
|---|---|
| Water | 5-10 |
| Glycerin | 5-10 |
| Starch | 30-35 |
| Alginate | 35-45 |
| Hyaluronic acid | 0.5-1.0 |
| Citrus Extracts | 2-4 |
| Fragrance, Colour | 1-2 |

### Example 6—Functional Layer Formulation (Face Mask)

The following table lists the ingredients of a functional layer formulation for a face mask to provide a blemish control function.

This particular functional layer formulation includes glycerin, starch and sodium alginate as film forming ingredients. The other listed ingredients except for colour and fragrance comprise the active ingredients of the functional layer. This functional layer formulation was applied to the base film formulation 3 listed in the table of Example 1 above, but may be applied to any of the base film formulations disclosed in Example 1.

| Ingredients | % Dry |
|---|---|
| Water | 5-10 |
| Glycerin | 5-10 |
| Starch | 30-35 |
| Alginate | 35-45 |
| Salicylic Acid | 2-5 |
| Tea tree oil | 0.5-1.0 |
| Chamomile | 0.5-1.0 |
| Fragrance, Colour | 2-4 |

### Example 7—Functional Layer Formulation (Insect Bite Patch)

The following table lists the ingredients of a functional layer formulation for a patch to provide relief from insect bites and/or stings.

This particular functional layer formulation includes glycerin, starch and sodium alginate as film forming ingredients. The other listed ingredients except for colour and fragrance comprise the active ingredients of the functional layer. This functional layer formulation may be applied to any of the base film formulations disclosed in Example 1.

| Ingredients | % Dry |
|---|---|
| Water | 5-10 |
| Glycerin | 5-10 |
| Starch | 30-35 |
| Alginate | 35-45 |
| Colloidal oatmeal | 3-7 |
| Tea tree oil | 0.05-0.1 |
| Extracts | 0.2-0.4 |
| Menthol | 0.2-0.5 |
| Fragrance, Colour | 0.5-1 |

### Example 8—Functional Layer Formulation (Insect Bite Patch)

The following table lists the ingredients of a functional layer formulation for a patch to provide relief from insect bites and/or stings.

This particular functional layer formulation includes glycerin, starch and sodium alginate as film forming ingredients. The other listed ingredients except for colour and fragrance comprise the active ingredients of the functional layer. This functional layer formulation may be applied to any of the base film formulations disclosed in Example 1.

| Ingredients | % Dry |
|---|---|
| Water | 5-10 |
| Glycerin | 5-10 |
| Starch | 30-35 |
| Alginate | 35-45 |
| Hydrocotisone | 0.5-1 |
| Chamomile | 0.05-0.1 |
| Fragrance, Colour | -1 |

### Example 9—Functional Layer Formulation (Face Mask or Patch)

The following table lists the ingredients of a functional layer formulation for a face mask or patch to provide a skin-tightening (anti-wrinkle) function.

This particular functional layer formulation includes glycerin, starch and sodium alginate as film forming ingredients. The other listed ingredients except for colour and fragrance comprise the active ingredients of the functional layer. This functional layer formulation may be applied to any of the base film formulations disclosed in Example 1.

| Ingredients | % Dry |
|---|---|
| Water | 5-10 |
| Glycerin | 5-10 |
| Starch | 10-15 |
| Alginate | 35-45 |
| Hydroxypropyl methylcellulose | 10-15 |
| Pullulan | 5-10 |
| Extracts | 0.4-1.5 |
| Fragrance, Colour | 1-2 |

### Example 10—Functional Layer Formulation (Patch)

The following table lists the ingredients of a functional layer formulation for a patch to provide an anti-cellulite function.

This particular functional layer formulation includes glycerin, carboxy methylcellulose, starch and sodium alginate as film forming ingredients. The other listed ingredients except for colour and fragrance comprise the active ingredients of the functional layer. This functional layer formulation may be applied to any of the base film formulations disclosed in Example 1.

| Ingredients | % Dry |
|---|---|
| Water | 5-10 |
| Glycerin | 5-10 |
| Carboxy Methyl Cellulose | 30-35 |
| Starch | 10-15 |
| Alginate | 20-30 |
| Caffeine | 0.5-1.0 |
| Citrus Aurantium Extract | 0.5-0.8 |
| Menthol | 0.2-0.5 |
| Vitamin C | 0.1-0.2 |
| Fragrance, Colour | 1-2 |

### Example 11—Functional Layer Formulation (Face Mask)

The following table lists the ingredients of a functional layer formulation for a face mask to provide a collagen firming function.

This particular functional layer formulation includes glycerin, carboxy methylcellulose, starch and sodium alginate as film forming ingredients. The other listed ingredients except for colour and fragrance comprise the active ingredients of the functional layer. This functional layer formulation may be applied to any of the base film formulations disclosed in Example 1.

| Ingredients | % Dry |
|---|---|
| Water | 5-10 |
| Glycerin | 5-10 |
| Carboxy Methyl Cellulose | 30-35 |
| Starch | 10-15 |
| Alginate | 20-30 |
| Hydrolyzed collagen protein | 3-4 |
| Hydrolyzed elastin | 1-2 |
| Vitamin C | 0.1-0.2 |
| Fragrance, Colour | 1-2 |

Although certain embodiments are described in the present disclosure, it is not restricted thereto. Rather, the disclosure includes all embodiments which may fall within the scope of the following claims.

What is claimed is:

1. A skin care product for topical application, comprising:
(a) a base layer comprising a flexible, biodegradable film, wherein the film comprises a biodegradable polymer matrix and a water-soluble plasticizer;
(b) a functional layer applied to one surface of the base layer, the functional layer comprising one or more active ingredients to provide a cosmetic and/or medicinal benefit when applied topically to a surface of a user's skin.

2. The skin care product of claim 1, wherein the biodegradable polymer matrix comprises one or more polymeric materials selected from the group comprising polysaccharides, flours, fibrous materials, gums and proteins.

3. The skin care product of claim 1, wherein the polymer matrix comprises about 35-80% of the base layer composition.

4. The skin care product of claim 1, wherein the biodegradable polymer matrix comprises one or more water soluble starches.

5. The skin care product of claim 4, wherein the each of the one or more water soluble starches is selected from the group comprising potato starch, wheat starch, tapioca starch, corn starch, rice starch, pea starch, bean starch, lentil starch, pulse starch and mung bean starch.

6. The skin care product of claim 1, wherein the biodegradable polymer matrix comprises one or more gums selected from the group comprising carrageenan, konjac, gellan gum, agar, guar gum, locust bean gum, xanthan gum, carboxymethylcellulose and alginates.

7. The skin care product of claim 1, wherein the biodegradable polymer matrix comprises one or more vegetable proteins selected from the group comprising soy protein, rice protein, pea protein, mung bean protein, hemp protein and pumpkin seed protein.

8. The skin care product of claim 1, wherein the biodegradable polymer matrix comprises one or more water-soluble starches and one or more gums, and wherein a weight ratio of starch:gum in the biodegradable polymer matrix is from about 2:1 to about 30:1.

9. The skin care product of claim 1, wherein the water-soluble plasticizer is glycerin, and wherein the base layer comprises about 10-30% of the plasticizer.

10. The skin care product of claim 1, wherein the base layer comprises about 10% water.

11. The skin care product of claim 1, wherein the base layer has a thickness of about 40-60 microns.

12. The skin care product of claim 1, wherein the active ingredients of the functional layer are selected from one or more of hyaluronic acid, ceramides, aloe vera, menthol, chamomile, pentavitin, niacinamide, peppermint oil, witch hazel, salicylic acid, tea tree oil, colloidal oatmeal, hydrocortisone, pullulan, caffeine, vitamin C, Paeonia suffruticosa root extract, Centella asiatica extract, and citrus extracts.

13. The skin care product of claim 1, wherein the skin care product is a face mask, and the active ingredients are selected from one or more of hyaluronic acid, ceramides, salicylic acid and natural extracts.

14. The skin care product of claim 1, wherein the skin care product is a cooling film, and the active ingredients are selected from one or more of menthol, aloe vera and natural extracts.

15. The skin care product of claim 1, wherein the skin care product is an insect bite film, and the active ingredients are selected from one or more of colloidal oatmeal, hydrocortisone and natural extracts.

16. The skin care product of claim 1, wherein the skin care product is for skin tightening, and the active ingredients are selected from one or more of hydroxypropyl methylcellulose, pullulan, hydrolyzed collagen, elastin, and natural extracts.

17. The skin care product of claim 1, wherein the functional layer further comprises one or more film forming compounds selected from a biodegradable polymer matrix and a water-soluble plasticizer.

18. The skin care product of claim 17, wherein the one or more film forming compounds are selected from the group comprising glycerin, water-soluble starch and alginate.

19. The skin care product of claim 1, wherein the functional layer has a thickness of about 35-40 microns.

20. The skin care product of claim 1, further comprising a third layer which is provided on top of the functional layer, such that the functional layer is sandwiched between the base layer and the functional layer, wherein the third layer has a non-aqueous composition.

21. The skin care product of claim 20, wherein the third layer comprises one or more oils selected from the group comprising coconut oil, avocado oil, and jojoba oil.

22. The skin care product of claim 20, wherein the third layer has a thickness of about 10-15 microns.
